(19) United States
(12) Patent Application Publication
(10) Pub. No.: US 2008/0188387 A1
(43) Pub. Date: Aug. 7, 2008

(54) Direct Assay of Skin Protein in Skin Samples Removed by Tape Stripping

(76) Inventors: Peter Horsewood, Dundas (CA); Robert Zawydiwski, Stoney Creek (CA)

Correspondence Address: BERESKIN AND PARR, 40 KING STREET WEST, BOX 401, TORONTO, ON, M5H 3Y2, omitted

(21) Appl. No.: 11/915,060

(22) PCT Filed: May 19, 2006
(86) PCT No.: PCT/CA06/00831

§ 371 Date: Nov. 20, 2007

Related U.S. Application Data
(60) Provisional application No. 60/682,837, filed on May 20, 2005.

(51) Int. Cl. A61K 8/00

(52) U.S. Cl. 510/130

(57) ABSTRACT

The present invention provides for a method of measuring the amount of skin removed by tape stripping. In one aspect of the invention, the invention provides a method for the direct assay of protein in skin samples removed by tape stripping, with a view to combining the protein measurement obtained with a corresponding skin cholesterol measurement to identify individuals at risk of having atherosclerosis as well as those at risk of developing atherosclerosis and similar diseases associated with and attributable to high cholesterol levels. Moreover, the present invention allows a comparative measurement of the amount of skin removed by tape stripping that does not rely solely on the area of the sample removed. Additionally, in one aspect of the invention, the method of this invention can allow relative levels of skin cholesterol to be compared based on the relative amounts of skin removed.

The section headings used herein are for organizational purposes only and are not to be construed as limiting the subject matter described in any way.

### FIELD

The present invention relates to a method of measuring the amount of skin removed by tape stripping. More particularly, the invention pertains to a method for the direct assay of protein in skin samples removed by tape stripping to determine a measurement of the amount of protein indicative of the amount of skin removed. Further, one aspect of the invention relates to a method for the direct assay of protein in skin samples removed by tape stripping with a view to combining the protein measurement obtained with a corresponding skin cholesterol measurement to identify individuals at risk of having atherosclerosis as well as those at risk of developing atherosclerosis and similar diseases associated with and attributable to high cholesterol levels.

### INTRODUCTION

As mentioned, one aspect of the invention relates to a method for the direct assay of protein in skin samples removed by tape stripping with a view to combining a protein measurement obtained with a corresponding skin cholesterol measurement to identify individuals at risk of having atherosclerosis as well as those at risk of developing atherosclerosis and similar diseases associated with and attributable to high cholesterol levels. Numerous studies have shown that atherosclerosis and its complications, such as heart attacks and strokes, are major causes of morbidity and mortality in almost all countries of the world.

Cost effective prevention of atherosclerosis requires the identification of individuals at risk, thereby allowing their medical treatment and change of life style. A desired goal is identifying those individuals belonging to the high-risk group but there are difficulties in selecting optimum methods for discriminating individuals at risk.

A widely used method for identifying individuals at risk of having atherosclerosis is based on the measurement of total cholesterol levels in venous blood plasma (Consensus Conference on Lowering Blood Cholesterol to Prevent Heart Disease, JAMA, 1985, 253, pg. 2080). Patients are considered to be at high-risk if their cholesterol level is over 240 mg/dL and there have been recent moves to lower this threshold level to lower values.

However, total cholesterol levels alone do not accurately predict a patient's risk level. A better prediction can be made by analyzing blood plasma lipoproteins; in particular, measurement of low density lipoprotein (LDL) and high-density lipoprotein (HDL) cholesterol levels is advantageous (Total and High Density Lipoprotein Cholesterol in the Serum and Risk of Mortality, British Medical Journal, 1985, 290, pg. 1239-1243).

Despite their advantage, use of the above methods requires blood sampling after a period of fasting. Additionally, the sampling is uncomfortable, poses a risk of infection and the required analysis of plasma lipoproteins and cholesterol is complicated and expensive. Moreover, studies have shown that blood plasma analysis may not entirely reflect the process of cholesterol accumulation in the arterial wall and other tissues. In many cases, neither plasma cholesterol levels nor even complete lipid profiles correlate with the severity of atherosclerosis.

Significant levels of cholesterol occur in tissue as well as in plasma and it has been shown that tissue cholesterol plays a leading role in development of atherosclerosis. Tissues, including skin, have been identified which accumulate cholesterol in the same way as the arterial wall and studies have demonstrated a close correlation between cholesterol content in the arterial wall and the skin. For example, cholesterol was extracted from lyophilized skin samples and measured using traditional chemical and biochemical techniques. (Nikitin Y. P., Gordienko I. A., Dolgov A. V., Filimonova T. A. “Cholesterol content in the skin and its correlation with lipid quotient in the serum in normals and in patients with ischemic cardiac disease”, Cardiology, 1987, II, No. 10, P. 48-51). While useful, this method is too complicated and painful to be employed for large scale population screening.

U.S. Pat. No. 4,458,686 describes a method of quantifying various compounds in the blood directly under the skin or on its surface. The method is based on measuring oxygen concentration changes electrochemically, for instance, via polarography. In the case of non-volatile substances that do not diffuse through the skin, it is necessary to implant enzymes under the skin to effect oxygen changes at the skin surface. This patent also discloses the potential of using such methods to quantify the amount of cholesterol using cholesterol oxidase. The complex instrumentation and procedures needed require the services of highly skilled personnel for making measurements, thus limiting the usefulness of the method for screening large numbers of people.

Determination of the cholesterol content in skin gives a measure of the extent of atherosclerosis and can be obtained through standard laboratory analysis of skin biopsy specimens. However, there is considerable pain involved in taking a skin sample and a risk of infection at the sampling site. In addition, this method has other disadvantages because the thick skin specimens incorporate several skin layers, including the outermost horny layer (stratum corneum), epidermis and dermis. Since the dermal layer is highly vascularized, skin biopsy samples contain blood vessels and blood elements. They may also contain sweat and sebaceous glands and the secretions contained therein. Additionally, subcutaneous fat is located directly under the derma and may also contaminate specimens. Therefore, skin biopsy specimens are heterogeneous and their analysis may give false data on cholesterol content in the skin.

U.S. Pat. No. 5,489,510 describes a non-invasive method for the visual identification of cholesterol on skin using a reagent having a specific cholesterol binding component in combination with a reagent having an indicator component to provide a visual color change corresponding to the presence of the component bound to cholesterol of the skin. The method overcomes many of the objections of earlier procedures and meets many of the desired goals required for a simple mass screening to identify individuals at risk of having atherosclerosis. The procedure is done directly on the palmar skin and, while it is quick and simple, it requires all individuals to be tested to be present at a doctor's office or clinic where the test is conducted. This of course limits effective large scale screening.

Molar ratios of the lipids, including cholesterol, in stratum corneum have been determined on samples obtained by direct, solvent extraction of skin (Norlen L., et al. J. Invest. Dermatology 72-77, 112, 1999). High performance liquid chromatography (HPLC) and gas liquid chromatography in conjunction with mass spectrometry were used to separate and analyze the lipids. The analytical methods are complex, but more importantly, the use of corrosive and irritant organic solvent systems to extract human skin for routine determinations is not practical.

The lipid profile of the stratum corneum layer of skin has been determined using a tape stripping method as described by A. Weerheim and M. Ponec (Arch. Dermatol. Res., 191-199, 293, 2001). In this study, lipids, including cholesterol, were solvent extracted from stratum corneum after tape stripping of skin. The resultant lipid extract was separated by high performance thin-layer chromatography. This method is very laborious. It requires three consecutive solvent systems to effect the separation of the lipids, a staining and charring method to visualize the components and a densitometry step to determine the relative amounts of the lipids. The method does not lend itself to the simple and rapid determination of cholesterol levels in large numbers of samples.

A device that provides a simple, high-throughput assay for measuring cholesterol on skin is an adhesive-tape device for skin-sampling and a novel method of its use, as disclosed in applicant's co-pending U. S. patent application, Publication No. US-2005-0272112-A1, the contents of the entirety of which are hereby incorporated by reference. The method disclosed in this application can be applied to obtain a number of skin samples from a number of individuals, so that skin cholesterol levels for the respective individuals can be made and their risk for atherosclerosis and related cardiovascular disease determined. The tape stripping devices and methods disclosed in this patent application provide for a simple, cost-effective risk assessment assay that is applicable to large-scale screening.

The total amount of cholesterol in a skin sample taken with an adhesive tape by tape stripping is related to the size of the tape sample. Therefore, to compare skin cholesterol levels between individuals, samples of the same size must be assayed and compared. This is achieved by using a tape stripping device that allows pieces of tape with a fixed area to be removed after applying the adhesive tape to the skin to obtain a sample. Some of the removed tape can be used for a skin cholesterol assay. For example, obtaining consistently sized skin samples from various individuals is accomplished by applying the adhesive tape repeatedly to the skin such that it becomes saturated with skin. Then, a small “dipstick” or “disk” having a fixed size is cut from the device to give a constant area of skin sample for the assay.

However, comparison of skin cholesterol levels between individuals using a constant area of tape saturated with skin does not necessarily assure that similar amounts of skin are being compared. Different total amounts of skin may be deposited onto respective tapes when taking samples from different individuals. By relating the skin cholesterol level to a standardized or normalized amount of skin would better allow comparison of skin cholesterol levels between individuals.

For example, one application of using a device for tape stripping to assay for measuring skin cholesterol is for individuals who apply for life insurance. Testing for risk factors (e.g., age, smoking status, blood pressure etc.) in insurance applicants is standard practice and allows premiums to be set based on testing results. Since skin cholesterol is a risk factor for having or developing atherosclerosis and similar diseases, its value could influence insurance premiums and samples could be subject to manipulation to favor outcome of results. Therefore, there is a requirement to ensure that an adequate skin sample has been taken for assaying skin cholesterol and thereby deter “cheaters” from deliberately under-sampling with the intention of producing a low skin cholesterol level. Consequently, for this application, there is a need to measure the amount of skin removed for the skin cholesterol assay to ensure that sufficient sample has been taken and to allow comparison of skin cholesterol levels between individuals for assessing risk status.

### SUMMARY

The present invention provides for a method of measuring the amount of skin removed by tape stripping. In one aspect of the invention, the invention provides a method for the direct assay of protein in skin samples removed by tape stripping, with a view to combining the protein measurement obtained with a corresponding skin cholesterol measurement to identify individuals at risk of having atherosclerosis as well as those at risk of developing atherosclerosis and similar diseases associated with and attributable to high cholesterol levels.

Moreover, the present invention allows a comparative measurement of the amount of skin removed by tape stripping that does not rely solely on the area of the sample removed. Additionally, in one aspect of the invention, the method of this invention can allow relative levels of skin cholesterol to be compared based on the relative amounts of skin removed.

It is also desirable that the method of measuring the amounts of skin samples removed by tape stripping should be simple, cost effective, amenable to high throughput processing, yet be compatible with methods involving, for example, and in accordance with one aspect of the invention, but not limited to, the assay of skin cholesterol in skin samples removed by tape stripping.

In particular, the invention comprises a method of measuring the amount of skin removed by tape stripping, comprising:

a) providing a tape having a backing member coated on at least one side thereof with a medical adhesive;

b) applying the tape onto a selected area of skin to adhere the tape to the selected skin area;

c) stripping the tape off the selected skin area to obtain a sample representative of an outer stratum corneum layer of the skin, the sample adhering to the tape so as to have exposed skin constituents;

d) applying a protein stain onto a predetermined surface area of the sample and allowing the protein stain to remain in contact therewith for a period of time sufficient to cause binding of said stain to protein present in the exposed skin constituents; and

e) measuring the stained protein in the exposed skin constituents to determine a measurement of the amount of protein indicative of the amount of skin removed.

The protein stain can be selected from the group consisting of anionic and acidic dyes. For one embodiment of the invention the protein stain is a Ponceau S stain reagent. For a further embodiment of the invention the protein stain is Coomassie Blue.

Moereover, the intensity of the stained protein in the exposed skin constituents is measured to determine a measurement of the amount of protein. In one embodiment the stained protein can be measured spectrophotometrically to determine a measurement of the amount of protein.

In a further embodiment the measurement of the amount of protein is compared to a predetermined threshold level. The sample can be discarded if the amount of protein measured is below the predetermined threshold.

Moreover, in another aspect of the invention the backing member can be formed of polyester.

In a further aspect of the invention the medical adhesive can be, for example, but not limited to, a pressure-sensitive adhesive; an acrylic based adhesive; a synthetic rubber elastomer adhesive; a silicone based adhesive; or comprises an elastomer formed of block polymers of styrene-isoprene-styrene or styrene-butadiene-styrene.

Moreover, a kit for use in carrying out the aforementioned method is also contemplated with this invention. The kit comprising:
the tape; and
a source of the protein stain.

The protein stain can be selected from the group consisting of anionic and acidic dyes. For one embodiment of the invention the protein stain is a Ponceau S stain reagent. For a further embodiment of the invention the protein stain is Coomassie Blue.

Moreover, in another aspect of the invention the backing member can be formed of polyester.

In a further aspect of the invention the medical adhesive can be, for example, but not limited to, a pressure-sensitive adhesive; an acrylic based adhesive; a synthetic rubber elastomer adhesive; a silicone based adhesive; or comprises an elastomer formed of block polymers of styrene-isoprene-styrene or styrene-butadiene-styrene.

Moreover, in another aspect of the invention, the adhesive is carried by a closeable device, the closeable device having a sampling member that carries the adhesive, and a closure member adapted to engage the sampling member and retain the adhesive within the device. The adhesive can be sealed within the device when the closure member engages the sampling member.

At least the closure member or the sampling member can be provided with a peripheral rim, and the other of the closure member or the sampling member can be provided with a peripheral groove adapted to receive the rim so that the adhesive is sealed within the device. Moreover, the closure member can be connected to the sampling member by a hinge.

Further, at least a portion of the sampling member can be adapted to be cut from the closeable device to form a dipstick, the dipstick having a first end thereof devoid of adhesive, and a second end thereof with adhesive. In another aspect of the invention at least a portion of the sampling member is adapted to be cut from the closeable device to form a disk, the disk having the adhesive provided on one face thereof. By using a dipstick having one end with adhesive, or a disk cut from the closeable device, a predefined and fixed area of the adhesive can be defined. Therefore, after sampling the skin, the dipsticks or disks have a skin sample attached to a definite and predefined area of adhesive.

Moreover, in further aspects of the invention, the sampling member of the closeable sampling device can have, for example, but not limited to, precut or pre-scored dipsticks or disks to predefine a fixed area of adhesive.

In addition, one aspect of the invention provides for a method of comparing the relative levels of skin cholesterol from a number of individuals, comprising:

a) measuring skin cholesterol from each individual by obtaining a sample representative of an outer stratum corneum layer of the skin using tape stripping and analyzing exposed skin constituents adhered to the adhesive to obtain a skin cholesterol level for an individual;

b) applying a protein stain onto a predetermined surface area of the exposed skin constituents of the sample separate from where the skin cholesterol is measured and allowing the protein stain to remain in contact therewith for a period of time sufficient to cause binding of said stain to protein present in the exposed skin constituents;

c) measuring the stained protein in the exposed skin constituents to determine a measurement of the amount of protein indicative of the amount of skin removed; and

d) normalizing the skin cholesterol measurement with the measurement of the amount of protein.

The normalizing can comprise dividing the skin cholesterol measurement by the protein measurement.

The protein stain can be selected from the group consisting of anionic and acidic dyes. For one embodiment of the invention the protein stain is a Ponceau S stain reagent. For a further embodiment of the invention the protein stain is Coomassie Blue.

Moereover, the intensity of the stained protein in the exposed skin constituents is measured to determine a measurement of the amount of protein. In one embodiment the stained protein can be measured spectrophotometrically to determine a measurement of the amount of protein.

In a further embodiment the measurement of the amount of protein is compared to a predetermined threshold level. The sample can be discarded if the amount of protein measured is below the predetermined threshold.

Moreover, in another aspect of the invention the backing member can be formed of polyester.

In a further aspect of the invention the medical adhesive can be, for example, but not limited to, a pressure-sensitive adhesive; an acrylic based adhesive; a synthetic rubber elastomer adhesive; a silicone based adhesive; or comprises an elastomer formed of block polymers of styrene-isoprene-styrene or styrene-butadiene-styrene.

These and other features of the applicant's teachings are set forth herein.

### DRAWINGS

The skilled person in the art will understand that the drawings, described below, are for illustration purposes only. The drawings are not intended to limit the scope of the applicant's teachings in any way.

FIG. 1 is a graph showing the chance of a particular sample having a selected reflectance value, taken for x1, x3, and, x10 stripped samples;

FIG. 2 is a graph showing the chance of a particular sample having an optical density (OD) 570 nm value taken for x1, x3, and, x10 stripped samples;

FIG. 3 is a top view of a sampling device as used in the Example;

FIG. 4 is a fragmentary view of the sampling device illustrated in FIG. 3, showing details of the sampling member thereof;

FIG. 5 is a perspective view of a dipstick cut from the sampling device of this invention;

FIG. 6 is a perspective view of an alternative sampling device that can be used in the Example; and

FIG. 7 is a perspective view of a disk cut from the sampling device of this invention from the alternative embodiment shown in FIG. 6.

### DESCRIPTION OF VARIOUS EMBODIEMNTS

A device that provides a simple, high-throughput, assay for measuring cholesterol on skin is an adhesive-tape device for skin-sampling, as disclosed in applicant's co-pending U.S. patent application, Publication No. US-2005-0272112-A1, the contents of the entirety of which are hereby incorporated by reference. In one aspect of the present invention, the method disclosed in this application can be applied to obtain a number of skin samples from a number of individuals, so that skin cholesterol levels for the respective individuals can be measured and their risk for atherosclerosis and related cardiovascular disease determined. The tape stripping devices and methods disclosed in this patent application can provide for a simple, cost-effective risk assessment assay that is applicable to large-scale screening.

For example, use can be made of a tape comprising a backing member formed of polyester. The tape is coated on at least one side thereof with a medical adhesive. The term “medical adhesive” as used herein refers to an adhesive which is hypoallergic and safe for application to the skin. Such an adhesive is preferably a pressure-sensitive adhesive, for example, an adhesive comprising an elastomer formed of block polymers of styrene-isoprene-styrene or styrene-butadiene-styrene.

As can be appreciated, there are many classifications and types of adhesives. In general, any adhesive suitable for use with this invention is a medical adhesive as defined above to ensure there will be generally no problems with allergic reactions when the adhesive was applied to the skin for sampling. The inventors tested several types of adhesives for use in taking a skin sample; the majority of these were pressure sensitive acrylic based adhesives, but several synthetic rubber type elastomer adhesives and silicone based adhesives were also tested.

The inventor has also found that synthetic rubber adhesives based on block copolymers of styrene and butadiene or styrene and isoprene perform well for this invention. An example of a synthetic rubber adhesive is a synthetic Kraton™ type adhesive (latex free) based on a block copolymer of styrene and butadiene. Such an adhesive provided better stability for skin samples to facilitate transportation of the samples for subsequent analysis.

A further preferred adhesive tape for use in the method of the invention is a double-coated pressure-sensitive medical grade tape. Examples of such a medical grade tape are those sold by 3M under Product #9877, or by Adhesive Research, Inc. under Product #8570.

A list of some of the other tapes that have been tested by the inventors is shown in the accompanying table. The one requirement that is constant is the use of a medical grade tape that is hypoallergenic.

TABLE 1

| Adhesive | Tape Product Name | Supplier |
|---|---|---|
| MA 27 Acrylic | AR 8570 | Adhesive Research, Inc. |
| MA 38 Acrylic | AR 7396 | Adhesive Research, Inc. |
| HY-3 Acrylic | AR 8311 | Adhesive Research, Inc. |
| Urethane liner | | |
| MA 65 Acrylic | AR 8944 | Adhesive Research, Inc. |
| MA 61 Acrylic | AR 8890 | Adhesive Research, Inc. |
| Acrylic | AR 8968 | Adhesive Research, Inc. |
| AS 124M Acrylic | AR 8651 | Adhesive Research, Inc. |
| Acrylic | MA 38 | Adhesive Research, Inc. |
| MA 31 Acrylic | MA 31 | Adhesive Research, Inc. |
| MA24 | MA 24A | Adhesive Research, Inc. |
| rosin tackified | | |
| polyisubutylene | | |
| Rubber solution | MA 70 | Adhesive Research, Inc. |
| Acrylic | MA 46 | Adhesive Research, Inc. |
| Acrylic | #888 | 3M |
| acid free | | |
| Silicone | N/A | Alza Corporation |
| Duragesic base | | |
| Silicone/acrylic | 702 | Scapa Group PLC |
| Silicone/silicone | 705 | Scapa Group PLC |

It can be appreciated that the adhesive tapes listed in Table 1 is not meant to be exhaustive, but merely illustrative of different adhesive tapes that are suitable for use with this invention at the present time, and that other adhesive tapes that will be apparent to those skilled in the art are contemplated by this invention.

Double-coated pressure-sensitive tapes are generally available with an easily removable protective liner. The liner protects the tape from adhering until it is removed and keeps the adhesive from becoming contaminated. Liners may be placed on either side of the double-coated tape or the tape may have a single liner and be wound onto itself, thereby protecting both surfaces.

Liners with differential release properties may be used so that a first side of adhesive may be exposed while protecting the second adhesive surface. A double-coated tape with differential liners is particularly advantageous for skin sampling. Removal of the first liner allows the tape to be stuck onto the backing support of a sampling device and leaves the skin-sampling side covered with the second liner. This second liner protects the skin sampling adhesive area from sticking and from contamination until it is to be used. When required for skin sampling, the second liner is removed.

The adhesive can be applied onto any part of skin, but the most suitable part is the surface of a palm because the palm does not have sebaceous glands whose secretions contain cholesterol that may affect results for certain aspects of this invention, and particularly those aspects involving measuring cholesterol. Additionally, the skin on the palm is readily accessible for sampling.

It is desirable to obtain uniform amounts of skin samples for analysis. Application of the adhesive for sampling is typically and routinely done using a single application of the adhesive to the skin. Additional amounts of stratum corneum material can be obtained by additional applications of the adhesive to the skin. Each subsequent application of the adhesive to the skin results in additional skin adhering to the adhesive. This process continues until the adhesive becomes saturated with skin material after which it is no longer sticky. The number of applications required to saturate an adhesive depends on the type of adhesive used, but for the most commonly used adhesives, saturation is achieved with less than ten applications, for example, but not limited to, three to seven applications. Applying adhesive to a fresh area of skin for each subsequent stripping results in better and faster saturation of the adhesive. Therefore, for consistent and good sampling, it is convenient to make ten applications of a adhesive to the skin, using new areas of skin for each application.

For those aspects of the invention, where skin cholesterol is being measured the total amount of cholesterol present in the skin sample on the adhesive is related to the size of the skin sample obtained. Moreover, a consistent skin sample size is required in order to compare relative levels of skin cholesterol between different individuals. Therefore, dipsticks can be cut from a device to present a fixed area of adhesive (for example, but not limited to, rectangular or circular area) that has exposed skin constituents attached thereto that were removed by the tape stripping. These dipsticks allow a comparison of levels of skin cholesterol between different individuals based on a fixed unit area of skin being analyzed.

As an alternative to cutting dipsticks from a device having a large area of tape or adhesive, the dipsticks can be, for example, but not limited to, precut or pre-scored to allow for easy separation from the device. Such precut or pre-scored dipsticks will, as described above, define a fixed and predefined area of tape or adhesive at one end thereof. After sampling, such a precut or pre-scored dipstick can then be easily removed from the device and contain an area of skin sample for analysis which is of a fixed and defined area.

Obtaining consistently sized skin samples from various individuals (or repeated samples from the same individual) is accomplished by the following steps. First, as previously described, the skin sample is taken by applying the adhesive repeatedly to the skin such that it becomes saturated with skin and is no longer sticky. The adhesive becomes saturated with skin after about three to seven applications and ten applications are routinely done to ensure saturation. Next, to obtain a constant area of skin sample to be assayed, a fixed sized area (as will be hereinafter become apparent from the Example) from the skin-sampling device is removed, and immersed in standardized volumes of detector and indicator reagents, as will also be described hereinafter.

The outer horny-layer of skin (stratum corneum) consists largely of protein-enriched corneocytes surrounded by a lipid mixture that includes cholesterol. Structurally, this is often depicted as a “bricks and mortar” model with the corneocytes representing the bricks and the surrounding lipid representing the mortar (P. M. Elias, J Invest Dermatol. 1983, 80, 44S-9S). The amount of protein in the stratum corneum is relatively constant between different individuals; therefore, protein in the skin sample removed by tape stripping can provide an indirect measure of the amount of skin removed. By measuring protein, and therefore the amount of skin sample obtained, it can then be determined, for one aspect of the invention, that an adequate skin sample has been removed for the skin cholesterol assay. Additionally, the skin cholesterol level measured can be compared with the protein value to obtain a measure of cholesterol per unit protein level and, thereby, cholesterol per unit amount of skin.

An assay to measure protein in the skin sample removed by tape stripping can use, for example, but not limited to, Coomassie Blue as a general protein stain (e.g., a protein stain commercially available as Bio Rad™). It has been shown that Coomassie Blue can be used for quantitative estimation of proteins immobilized onto a support material (S. Fazekas de St. Groth, et al. Biochimica Et Biophysica Acta, 1863, 71, 377-391). The Coomassie stain is applied to an area of skin on the adhesive and after a suitable staining period the excess stain is washed away. The intensity of stained skin protein is then determined by measuring the chroma of the blue stained sample on the adhesive and this intensity is related directly to the amount of protein. Measuring the relative intensity (e.g., chroma) of the stained skin samples allows relative amounts of skin samples taken from individuals by tape stripping to be compared.

The following measurements were taken to establish how the number of tape strippings correlate with the amount skin removed, as measured using Bio-Rad protein staining. In accordance with one aspect of the invention, these measurements were made to determine if removal of an inadequate amount of skin might affect the skin cholesterol test. Therefore, assays were run to measure the protein removed with variable numbers of tape stripping. In particular, palms of volunteers were stripped one (1), three (3) and ten (10) times and the protein levels determined by reading chroma and reflectance at 610 nm after staining with Bio-Rad protein dye reagent. These protein levels provided a measure of the extent of skin removal and the efficiency of tape stripping.

In the experiment, Bio-Rad protein determinations were done on samples collected from forty-five (45) volunteers. Each volunteer provide skin samples taken using one (1), three (3) and ten (10) tape strippings.

For the three different sample groups (labeled x2, x3 and x10) it was found that there was good correlation between chroma and reflectance at 610 nm. From a linear least squares regression analysis the R2 values for the x1, x3 and x10 samples were 0.961, 0.978 and 0.959 respectively. The mean chroma values for the x1 stripped samples (n=45) was 11.07, sd 1.83, CV16.3%; the mean chroma values for the x3 stripped samples (n=44) was 15.46, sd 2.32, CV17.3%; and the mean chroma values for the x10 stripped samples (n=44) was 23.50, sd 1.06, CV4.6%. The mean reflectance values for the x1 stripped samples (n=45) was 55.69, sd 2.95, CV5.6%; the mean reflectance values for the x3 stripped samples (n=44) was 48.07, sd 3.08, CV6.4%; and the mean reflectance values for the x10 stripped samples (n=44) was 36.19, sd 1.48, CV4.1%. There were significant differences between each of the groups.

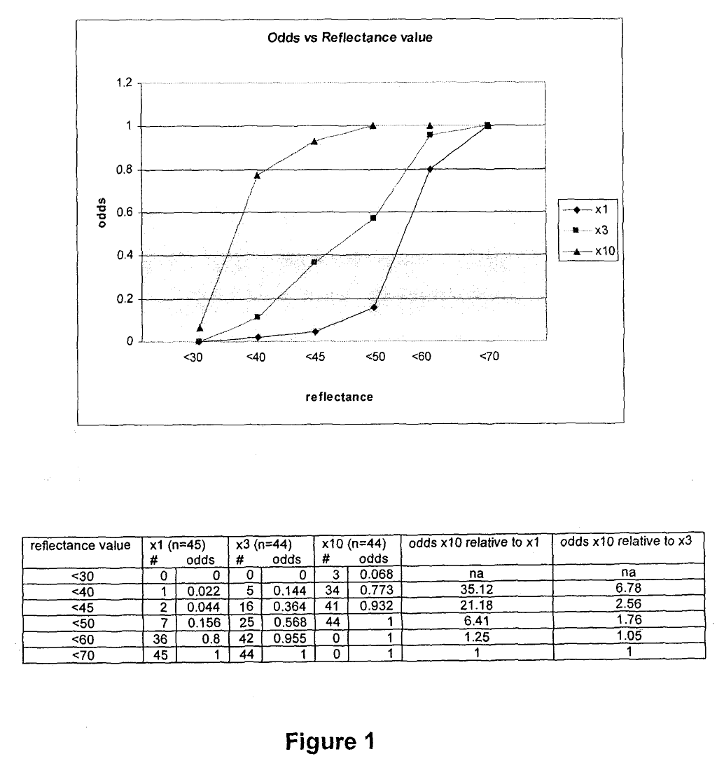

An analysis for the chance of a particular sample having a certain reflectance (610 nm) value were done for each group and are shown in FIG. 1. This analysis indicated that 93.2% of the x10 stripped samples had a reflectance value of <45, 36.4% of the x3 stripped samples had a reflectance value of <45 and only 4.4% of the x1 stripped samples had a reflectance value of <45. Alternatively, 77.3% of the x10 stripped samples had a reflectance value of <40, 11.4% of the x3 stripped samples had a reflectance value of <40 and only 2.2% of the x1 stripped samples had a reflectance value of <40.

It was found that using a cut-off value of <45 as acceptance criteria for adequate stripping would require rejection of 6.8% of the samples that had been obtained using ten times strippings. A cut-off value of <45 would result in the rejection of 63.6% of samples that had been obtained using three times strippings and rejection of 95.6% of samples that had been obtained with just a single stripping.

While the Coomassie Blue staining method works well, it requires a reflectance spectrometer able to read chroma values. Additionally, it requires each stained sample to be read individually, whereas the skin cholesterol assay can use 96 well micro plates to be amenable to processing large numbers of samples.

An assay has been developed that allows the amount of protein on a skin sample to be determined easily, and, on many skin samples simultaneously, if desired. Moreover, the assay allows protein to be measured using readily available spectrophotometers, for example, 96 well-reading spectrophotometers, in place of single sample chroma measurements. This assay is based on Ponceau S staining (i.e., a member of the general class of anionic or acidic dyes that binds to proteins, for example, but not limited to, basic amino acid residues).

Ponceau S stain binds to proteins and is used in histochemical staining of tissues and it is also used as a reversible membrane stain for nitrocellulose bound proteins (O. Salinovich and R. C. Montelaro, Anal. Biochem. 1986, 156, 341-347). This method allows stained proteins to be visualized on membranes and later, after the stain is removed, permits the proteins to be characterized further without interference from any bound stain. For the current invention, the Ponceau S method is further developed, as will hereinafter be explained, and shows that skin samples taken by tape stripping can be stained and, after washing the skin sample, the stain can be eluted and quantified spectrophotometrically. Ponceau S stains keratin particularly well and keratin is the main protein in the horny layer of skin that is removed by tape stripping.

Despite the fact that ten applications results in saturation of the adhesive (i.e., loss of adhesion), results have shown that different individuals give different amounts of skin. Although individuals may have slightly different levels of keratin and other skin proteins the inventors believe the differences seen are unlikely due to this. For example, different individuals may show several fold differences in measured protein levels, but protein levels of corneocytes would not be expected to vary by this amount for the skin samples taken in accordance with this invention. The inventors believe that the differences are more likely due to different total amounts of skin that are removed during tape stripping. This is suggested since some individuals seem to saturate the adhesive after only two to four strippings, whereas others require six to eight strippings to saturate the adhesive: this implies different amounts of skin are removed.

In one aspect of the invention, skin cholesterol measurements taken by tape stripping are often used to determine risk of coronary artery disease and related adverse thrombotic events and this finds particular use in the insurance industry. Individuals seeking life insurance are assessed for various risk factors (e.g., age, smoking status, blood pressure etc.) and premiums are based on total risk. Since skin cholesterol is a risk factor, its value could affect insurance premiums and samples could be subject to manipulation to favor outcome of results. Therefore, further advantages of determining protein levels is to ensure that an adequate sample has been taken (i.e., above a pre-selected threshold) and thereby deter “cheaters” from deliberately under-sampling with the intention of producing a low skin cholesterol level.

To ensure that an adequate skin sample is taken for skin cholesterol measurements, a minimum threshold level of skin protein is set. The threshold level is set by analyzing many skin samples and determining the distribution range of protein values. Distribution ranges for skin samples taken with a single tape stripping or with multiple tape strippings including saturation stripping (i.e., ten tape strippings) are prepared. From these distribution ranges a threshold limit can be chosen so that the likelihood can be determined that a protein value for a particular sample is within the limits of a pre-defined population. Ideally, there would be a threshold value that would allow all samples taken with ten tape strippings to be completely distinguished from samples taken with a single tape stripping, and possibly also from samples with, for example, three tape strippings. In actual practice the distribution curves overlap and it appears from the nature of an individual's variability to release skin on tape stripping, that complete discrimination is not possible. Nevertheless, threshold values can be chosen that allow a high percentage of the ten tape stripped samples to be readily distinguished from most of the samples taken with the single tape stripping.

For example it is useful to choose a threshold level, based on ten tape strippings, such that 98% of the population have values above that level, then on average two samples of each one hundred analyzed will be rejected as having insufficient sample. However, this threshold level will ensure that the majority of samples taken with a single tape stripping will not have sufficient protein and will be rejected. These tape strippings that give values below the pre-selected threshold level are deemed to have an insufficient skin sample for reliable skin cholesterol determinations.

To establish how the number of tape strippings correlates with the amount skin removed as measured by Ponceau S protein staining, the following experiment was undertaken. To see how the amount of skin removed varies with the number of tape strippings made, skin samples were removed from palms of 50 volunteers (N=50) by stripping one (1), three (3), and ten (10) times and the protein levels determined after staining with Ponceau S dye solution. The protein levels were based on optical density readings of dye eluted from the stained skin samples.

The results were analyzed to determine if the values were normally distributed and to determine if cut-off values can be established to differentiate samples that were obtained using a particular number of tape strippings. Samples for x1, x3 and x10 strippings were obtained from 50 volunteers (the results reflect that complete data was available for 49 samples only).

The mean optical density for the x1 strippings (N=50) was 0.078 with a mean CV of 13.0%; the mean optical density for the x3 strippings (N=50) was 0.131 with a mean CV of 12.3%; and the mean optical density for the x10 strippings (N=49) was 0.263 with a mean CV of 12.0%.

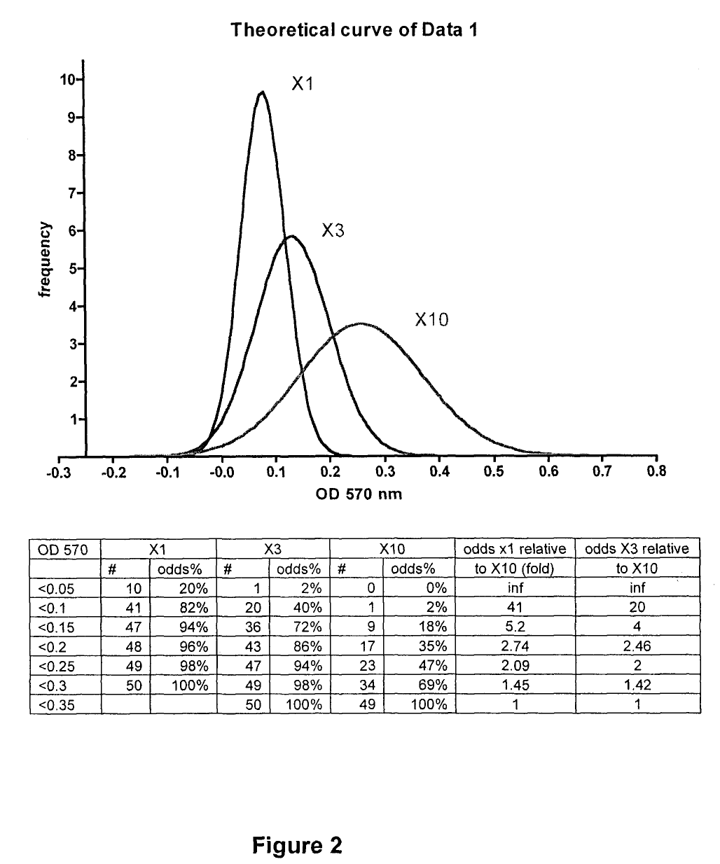

Analysis using the Anderson-Darling or Shapiro-Wilk test indicated a non-normal distribution for the data. Analysis for the chance of a particular sample having an optical density (OD) 570 nm value were done for each group and are shown in FIG. 2.

This analysis indicated that 98% of the x10 stripped samples had an OD 570 nm>0.1; 60% of the x3 stripped samples had an OD 570 nm>0.1; and only 18% of the x1 stripped samples had an OD 570>0.1.

Therefore, choosing a cut-off value of >0.1 OD as a criteria for sufficient sampling will result in rejection of 2% of the x10 stripped samples, 40% of the x3 stripped samples, and 82% of the x1 stripped samples.

The protein level may also be used to normalize skin cholesterol levels that vary as a result of variable skin samples. For instance, individuals who give a small skin sample but who have high skin cholesterol level may give a skin cholesterol value that is similar to an individual who has low skin cholesterol levels but gives a large skin sample. By normalizing to a constant protein level the two individuals may be distinguished as having high and low skin cholesterol respectively. The normalization could involve the simple manipulation of dividing the cholesterol level by the protein level; this effectively gives the cholesterol level per unit protein.

### EXAMPLE

Aspects of the applicant's teachings may be further understood in light of the following example, which should not be construed as limiting the scope of the present teachings in any way.

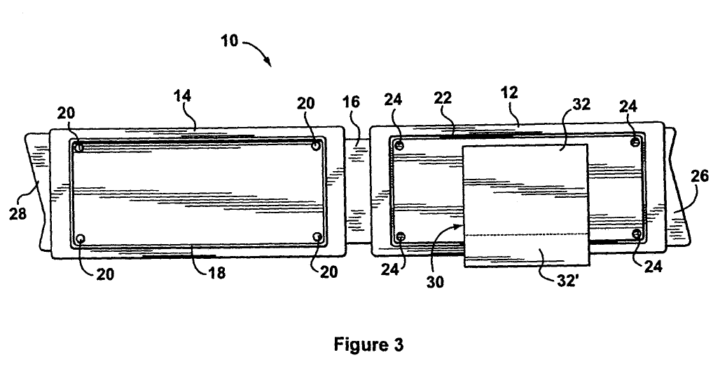

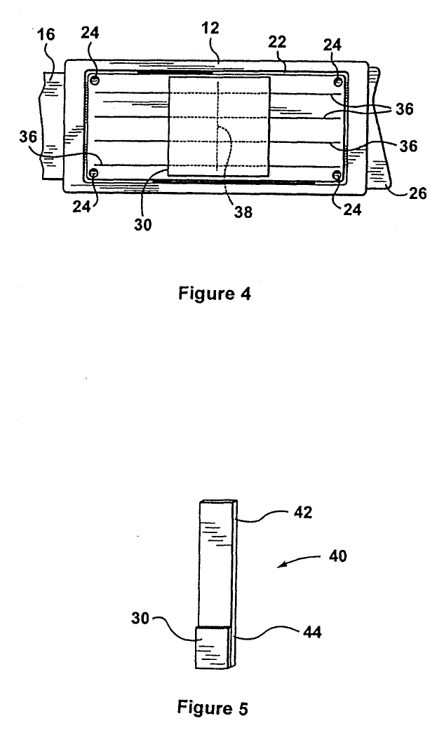

Use was made of a sampling device as shown in FIG. 3. The sampling device, which is generally designated by reference numeral 10, is formed of plastic (polypropylene) and comprises a sampling member 12 connected to a closure member 14 by an integral hinge 16. The closure member 14 has a peripheral rim 18 and four pins 20, adapted to lock into, respectively, a peripheral groove 22 and four holes 24 formed in the sampling member 12. Folding the hinge 16 causes engagement of the rim 18 with the groove 22 and of the pins 20 with the holes 24, thereby ensuring that the two halves of the device 10 remain closed and sealed to prevent dust and contamination of the interior surfaces. The outer surface (not shown in FIGS. 3 and 4) of the closure member 14 has a flat area for receiving a label and barcode strip, for sample identification. The sampling member 12 and closure member 14 are respectively provided with finger-tabs 26 and 28 for opening the device 10.

A double-coated pressure-sensitive medical grade tape 30 having a protective Kraft paper release liner 32 and sold by 3M under Product #9877 was adhered to the central area of the sampling member 12. The release liner 32 is wider than the adhesive tape 30, thereby defining a strip 32′ along one edge with no attached tape. This strip 32′ of liner overhangs the edge of the device to form a tab for easy removal of the liner. Immediately before use, the liner 32 is removed using the overhanging tab 32′ and this exposes the adhesive of the tape 30 for skin sampling.

The palmar skin area for sampling was cleaned and dried. The tape 30 with the exposed adhesive was applied onto the palm. The tape 30 was pressed against the skin by applying pressure to the back of the sampling member 12 above the adhesive area, thereby causing adherence of the stratum corneum layer. The device 10 was peeled away, reapplied to a new area of the palm and again pressed to the skin. The device was peeled away and applied to the palmar skin in this way for a total of 10 applications.

A dipstick 40 (see FIG. 5) about five mm in width is cut from the device 10 after application to the skin, as follows. Referring to FIG. 4, an end portion of the sampling member 12 was removed by cutting along the portion of groove 22, which is adjacent to the tab 26. Three cuts were then made along guide lines 36 (shown in FIG. 4) molded into the sampling member 12, to delineate the five mm stick, cutting from the edge to just past the centre line. The 5 mm wide stick was released from the sampling member 12 by making a third cut across the center of the member 12, using guideline 38 molded into the member 12. Stick 40 has a first end portion 42 devoid of tape and a second end portion 44 with tape having the skin sample adhered thereto.

As an alternative to cutting dipsticks 40 from device 10 having a large area of tape or adhesive, the dipsticks 40 can have guide lines 36 and 38 precut or pre-scored to allow for easy separation of the dipsticks 40 from the device 10. The dipsticks, whether cut, precut, or pre-scored, will define a fixed and predefined area of tape or adhesive at the second end portion 44. Therefore, after sampling, dipstick 40 is removed from the device 10 and contains an area of skin sample for analysis that is of a fixed and defined area.

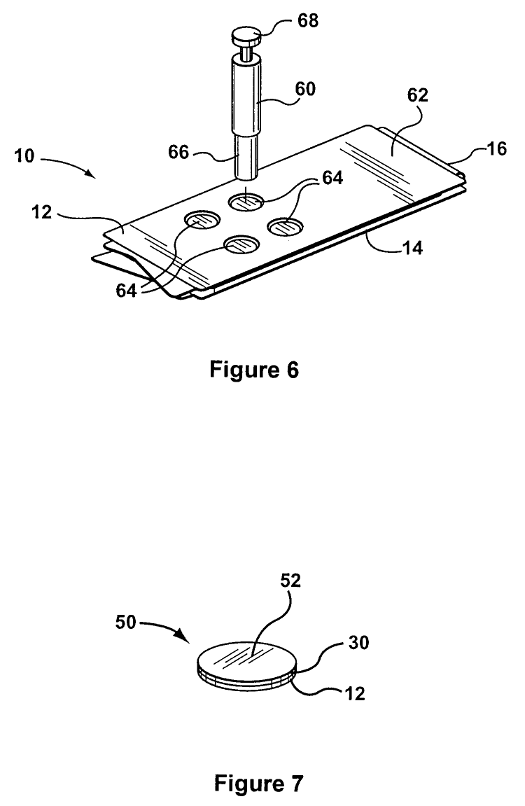

As an alternative to dipstick 40, FIG. 6 shows a cutting tool 60 that can be used to remove a disk 50, as illustrated in FIG. 7. Disk 50 has skin samples adhered to the adhesive tape 30 from the sampling device 10 on one face 52 thereof. Cutting tool 60 can remove a disk from the device 10 when the device 10 is in a folded over (closed) position, as illustrated. The closed device is placed on a firm surface (not illustrated) with the outer surface 62 of the sampling member 12 of the device 10 facing up. The cutting tool 60 is inserted in a circular depression 64 that can be provided on the outer surface 62 of the sampling member 12 of device 10 and the cutting tool 60 is then pressed down to cut through the plastic and the tape 30/skin sample. The cutting tool 60 is not pressed down so far, however, so as to cut through the plastic of the closure member 14 of the device 10.

As an alternative to cutting disks 50 from device 10 disks 50 can be precut or pre-scored (for example, but not limited to, at the boundary of the circular depression), to allow for easy separation of the disks 50 from the device 10. As with the dipsticks, however, the disks, whether cut, precut, or pre-scored, will define a face 52 of fixed and predefined area of tape or adhesive. Therefore, after sampling, disk 50 is removed from the device 10 and contains an area of skin sample for analysis that is of a fixed and defined area.

Continuing the Example with the dipstick 40 from FIG. 5, the dipstick to be assayed was placed into approximately 150 μL solution of Ponceau S stain reagent (for example, product P7170 as provided by Sigma-Aldrich Canada Ltd.) in a well of a 96 well microwell plate (not illustrated). The stick was left in the solution for about fifteen minutes at room temperature, after which it was removed and placed into a new well of a microwell plate containing approximately 200 μL of water wash-solution. The microwell plate was agitated to effect washing and after about one minute the stick was removed to a new well containing approximately 200 μL of fresh water wash-solution and again agitated for about one minute. Washing with agitation was done a third time. After the third wash any droplets of wash solution on the bottom of the stick were removed by gently blotting on a blotting tissue, which was placed on a clean flat surface.

Bound stain reagent was then eluted from the stick by placing it into a well containing approximately 150 μL of 0.1 N sodium hydroxide solution. After agitation of the stick in the microwell for about 5 minutes the stick was removed and the amount of eluted stain determined by measuring the absorbance of the solution at 550-570 nm.

To allow many samples to be processed together requires that the dipsticks 40 be held in a configuration that matches that of a standard 96 well (8×12) microplate. Instruments are available that can dispense reagents into these plates and also to wash the wells, a requirement that is necessary to prevent reagent carry-over between assay steps. Spectrophotometers that can read the coloured solutions directly in the wells at the final step of the assay are also readily available. This was achieved using customized fixtures that hold up to 96 dipsticks in the correct orientation and position so that they fit into the wells of a standard 96 well microplate The fixtures allow the group of sticks, up to 96, to be removed together as a group to new wells at each step of the assay. The group of sticks is then processed in a procedure using the same reagents and method as described above for the single stick assay.

In addition to the protein measurement above, for one aspect of the invention, additional dipsticks can be cut from the device to determine the amount of skin cholesterol. For measuring the amount of skin cholesterol the dipsticks were each placed into approximately 100 μL solution of an A-C-B reagent in wells of a 96 well microwell plate (not illustrated). The reagent was a conjugate of digitonin (A) linked to horseradish peroxidase (B) through a maleic anhydride-N-vinylpyrrolidone copolymer (C) and was used at a concentration of approximately 1 μg/mL. The sticks were left in the solution for about fifteen minutes at room temperature, after which they were removed and placed into new wells of a microwell plate containing approximately 200 μL of wash solution. The microwell plate was agitated to effect washing and after about one minute the sticks were removed to new wells containing approximately 200 μL of fresh wash solution and again agitated for about one minute. Washing with agitation was done a third time, after which the sticks were removed and placed in approximately 100 μL of a substrate solution (Enhanced K-Blue reagent). The sticks were then incubated with the substrate solution, in the dark, for about fifteen minutes at room temperature. The microwell plate can be shaken during this step.

After the sticks were incubated, the sticks can then be removed. Approximately one hundred (100) μL of 1 N sulfuric acid is then added to the wells with the substrate solution to stop further reaction, and the optical density of the resulting solution was read at about 450 nm on a plate reading spectrophotometer, to provide a measure of the amount of cholesterol in the skin sample.

By measuring the protein levels and the amount of skin cholesterol from a sampling device as described, allows the cholesterol measurement to be cross-referenced to the respective protein level, to determine, for example, whether enough of a skin sample has been obtained to provide a useable skin cholesterol measurement. Alternatively, or in addition thereto, the protein level can be used to normalize the skin cholesterol levels, such as, for example, by dividing the cholesterol level of one person by the protein level.

While the applicant's teachings are described in conjunction with various embodiments, it is not intended that the applicant's teachings be limited to such embodiments. On the contrary, the applicant's teachings encompass various alternatives, modifications, and equivalents, as will be appreciated by those of skill in the art.

1. A method of measuring the amount of skin removed by tape stripping, comprising:
a) providing a tape having a backing member coated on at least one side thereof with a medical adhesive;
b) applying the tape onto a selected area of skin to adhere the tape to the selected skin area;
c) stripping the tape off the selected skin area to obtain a sample representative of an outer stratum corneum layer of the skin, the sample adhering to the tape so as to have exposed skin constituents;
d) applying a protein stain onto a predetermined surface area of the sample and allowing the protein stain to remain in contact therewith for a period of time sufficient to cause binding of said stain to protein present in the exposed skin constituents; and
e) measuring the stained protein in the exposed skin constituents to determine a measurement of the amount of protein indicative of the amount of skin removed.

2. A method as claimed in claim 1, wherein said protein stain is selected from the group consisting of anionic and acidic dyes.

3. A method as claimed in claim 1, wherein said protein stain is a Ponceau S stain reagent.

4. A method as claimed in claim 1, wherein said protein stain is Coomassie Blue.

5. A method as claimed in claim 1, wherein the intensity of the stained protein in the exposed skin constituents is measured to determine a measurement of the amount of protein.

6. A method as claimed in claim 1, wherein the stained protein is measured spectrophotometrically to determine a measurement of the amount of protein.

7. A method as claimed in claim 1, wherein the measurement of the amount of protein is compared to a predetermined threshold level.

8. A method as claimed in claim 7, wherein the sample is discarded if the amount of protein measured is below the predetermined threshold.

9. A method as claimed in claim 1, wherein said backing member is formed of polyester.

10. A method as claimed in claim 1, wherein said medical adhesive is a pressure-sensitive adhesive.

11. A method as claimed in claim 1, wherein said medical adhesive is an acrylic based adhesive.

12. A method as claimed in claim 1, wherein said medical adhesive is a synthetic rubber elastomer adhesive.

13. A method as claimed in claim 1, wherein said medical adhesive is a silicone based adhesive.

14. A method as claimed in claim 1, wherein said medical adhesive comprises an elastomer formed of block polymers of styrene-isoprene-styrene or styrene-butadiene-styrene.

15. A method of comparing the relative levels of skin cholesterol from a number of individuals, comprising:
a) measuring skin cholesterol from each individual by obtaining a sample representative of an outer stratum corneum layer of the skin using tape stripping and analyzing exposed skin constituents adhered to the adhesive to obtain a skin cholesterol level for an individual;
b) applying a protein stain onto a predetermined surface area of the exposed skin constituents of the sample separate from where the skin cholesterol is measured and allowing the protein stain to remain in contact therewith for a period of time sufficient to cause binding of said stain to protein present in the exposed skin constituents;
c) measuring the stained protein in the exposed skin constituents to determine a measurement of the amount of protein indicative of the amount of skin removed; and
d) normalizing the skin cholesterol measurement with the measurement of the amount of protein.

16. A method according to claim 15, wherein the normalizing comprises dividing the skin cholesterol measurement by the protein measurement.

17. A method as claimed in claim 15, wherein said protein stain is selected from the group consisting of anionic and acidic dyes.

18. A method as claimed in claim 15, wherein said protein stain is a Ponceau S stain reagent.

19. A method as claimed in claim 15, wherein said protein stain is Coomassie Blue.

20. A method as claimed in claim 15, wherein the intensity of the stained protein in the exposed skin constituents is measured to determine a measurement of the amount of protein.

21. A method as claimed in claim in claim 15, wherein the stained protein is measured spectrophotometrically to determine a measurement of the amount of protein.

22. A method as claimed in claim 15, wherein the measurement of the amount of protein is compared to a predetermined threshold level.

23. A method as claimed in claim 22, wherein the sample is discarded if the amount of protein measured is below the predetermined threshold.

24-29. (canceled)

30. A kit for use in carrying out a method as defined in claim 1, comprising:
said tape; and
a source of said protein stain.

31. A kit as claimed in claim 30, wherein said protein stain is selected from the group consisting of anionic and acidic dyes.

32. A kit as claimed in claim 30, wherein said protein stain is a Ponceau S stain reagent.

33. A kit as claimed in claim 30, wherein said protein stain is Coomassie Blue.

34-39. (canceled)

40. A kit as claimed in claim 30, wherein said adhesive is carried by a closeable device, the closeable device having a sampling member which carries the adhesive, and a closure member adapted to engage the sampling member and retain the adhesive within the device.

41. A kit as claimed in claim 40, wherein said adhesive is sealed within the device when the closure member engages the sampling member.

42. A kit as claimed in claim 41, wherein the at least the closure member or the sampling member is provided with a peripheral rim, and the other of the closure member or the sampling member is provided with a peripheral groove adapted to receive the rim so that the adhesive is sealed within the device.

43. A kit as claimed in claim 40, wherein the closure member is connected to the sampling member by a hinge.

44. A kit as claimed in claim 40, wherein at least a portion of the sampling member is adapted to be cut from the closeable device to form a dipstick, said dipstick having a first end thereof devoid of adhesive, and a second end thereof with adhesive.

45. A kit as claimed in claim 40, wherein the closeable device defines at least one dipstick therein that is precut or pre-scored, the at least one dipstick having a first end thereof devoid of adhesive and a second end thereof with adhesive.

46. A kit according to claim 44, wherein the second end of the dipstick with adhesive defines a fixed and predefined area of adhesive.

47. A kit as claimed in claim 40, wherein at least a portion of the sampling member is adapted to be cut from the closeable device to form a disk, said disk having the adhesive provided on one face thereof.

48. A kit as claimed in claim 40, wherein the closeable device defines at least one disk therein that is precut or pre-scored, the at least one disk having the adhesive on one face thereof.

49. A kit according to claim 47, wherein the face of the disk with adhesive defines a fixed and predefined area of adhesive.
